(19) United States
(12) Patent Application Publication
(10) Pub. No.: US 2025/0177421 A1
(43) Pub. Date: Jun. 5, 2025

(54) METHOD FOR PREVENTING OR TREATING HEARING LOSS

(71) Applicant: MACKAY MEDICAL COLLEGE, New Taipei City (TW)

(72) Inventor: YI-CHAO HSU, New Taipei City (TW)

(73) Assignee: MACKAY MEDICAL COLLEGE, New Taipei City (TW)

(21) Appl. No.: 18/843,652

(22) PCT Filed: Mar. 6, 2023
(86) PCT No.: PCT/US2023/063756

§ 371 Date: Sep. 3, 2024

Related U.S. Application Data
(60) Provisional application No. 63/316,894, filed on Mar. 4, 2022.

(51) Int. Cl. A61K 31/6615; A61K 31/336; A61P 27/16

(52) U.S. Cl. CPC A61K 31/6615; A61K 31/336; A61P 27/16

(57) ABSTRACT

Provided is a method for preventing or treating hearing loss in a subject in need thereof. The method includes administering to the subject an effective amount of inositol hexaphosphate or a salt or derivative thereof, fucoxanthin or a derivative thereof, or a combination thereof. Also provided is a composition for use in preventing or treating hearing loss in a subject in need thereof.

### BACKGROUND

### 1. Technical Field

The present disclosure relates to prophylaxis or treatment of hearing loss, particularly to methods for preventing or treating hearing loss by administering to a subject in need thereof a composition comprising inositol hexaphosphate or a salt or derivative thereof, fucoxanthin or a derivative thereof, or a combination thereof. Also related is a composition for use in preventing or treating hearing loss in a subject in need thereof.

### 2. Description of Related Art

One of the most common types of hearing loss is sensorineural hearing loss (SNHL) (also known as sensorineural deafness) that is caused by the loss of hair cells and/or auditory spiral ganglion neurons or their functions and leads to communication difficulties in a large percentage of the population. Hair cells are sensory cells in the cochlea responsible for transduction of sound into an electrical signal. The human inner ear contains about 15,000 hair cells per cochlea at birth, while these cells do not regenerate after birth and may be lost as a result of various genetic or environmental factors (e.g., noise exposure, mechanical injury, ototoxic drug toxicity (e.g., cisplatin and aminoglycosides), viral infection (e.g., otitis media), aging, and genetic defects).

Auditory dysfunction in humans typically arises from aging or both acute and chronic exposures to loud sounds or ototoxic chemicals. Sounds exceeding 85 decibels can cause hearing loss and are generated by sound sources, such as gun shots, exploding bombs, jet engines, power tools, and musical concerts. Other common daily activities and products also give rise to high intensity noise, such as the use of hair dryers, lawn mowers, and blenders. Side effects of noise-induced hearing loss include tinnitus, diminished speech understanding, hyperacusis, loudness recruitment and various types of auditory processing impairments. In addition, exposures to commonly used medications may also induce auditory dysfunctions. For example, patients treated with chemotherapy, antibiotics and other medications often develop hearing loss as a side effect. Furthermore, auditory dysfunction is also a common consequence of aging.

Currently, very few cases of hearing loss can actually be cured. Audio logical devices such as hearing aids mainly amplify sound and cannot correct for suprathreshold or retrocochlear impairments, and thus have limitations including the inability to improve speech intelligibility, tinnitus, hyperacusis, loudness recruitment and various other types of central auditory processing disorders. On the other hand, in cases of age-related, noise- or drug-induced auditory dysfunctions, the effective way to “treat” the disorders or reduce its severity is prevention, e.g., avoiding excessive noise and using ear protectors, practicing a healthy lifestyle, and avoiding exposure to ototoxic drugs and substances if possible.

Hair cells are also found in the utricle of the vestibule, an organ which regulates balance. Therefore, there is an unmet need for protecting or promoting the function of hair cells before or after injury, so as to restore hearing and vestibular function.

### SUMMARY

In view of the foregoing, it was surprisingly found that inositol hexaphosphate, also known as phytic acid or IP6, when administered, is capable of improving the loss of hair cells, representing an alternative strategy for prevention or treatment of hearing loss. Additionally, it was also found that fucoxanthin may protect the cochlear hair cells and reduce the hearing thresholds, and the combination of inositol hexaphosphate and fucoxanthin even has a synergistic effect in preventing or treating hearing loss.

In at least one embodiment of the present disclosure, a composition for preventing or treating hearing loss is provided. The composition comprises at least one of inositol hexaphosphate or a salt or derivative thereof and fucoxanthin or a derivative thereof, and at least one of a pharmaceutically acceptable excipient thereof and a food excipient thereof. In some embodiments, the composition may comprise a combination of the inositol hexaphosphate or the salt or derivative thereof and the fucoxanthin or the derivative thereof.

In at least one embodiment of the present disclosure, the salt or derivative of the inositol hexaphosphate is selected from the group consisting of sodium phytate, potassium phytate, calcium phytate, dipotassium phytate, calcium-magnesium phytate, magnesium phytate, and sodium phytate dodecahydrate.

In at least one embodiment of the present disclosure, the derivative of the fucoxanthin may be fucoxanthinol, iso-fucoxanthin, iso-fucoxanthinol, or amarouciaxanthin A.

In at least one embodiment of the present disclosure, the inositol hexaphosphate or the salt or derivative thereof and the fucoxanthin or the derivative thereof are independently present in an amount of from 0.1% to 60% of the composition by weight, such as 0.1%, 0.2%, 0.3%, 0.4%, 0.5%, 0.75%, 1%, 2%, 5%, 10%, 15%, 20%, 25%, 30%, 35%, 40%, 45%, 50%, 55%, and 60%. In some embodiments, an amount of the inositol hexaphosphate or the salt or derivative thereof or the fucoxanthin or the derivative thereof in the composition has a lower limit chosen from 0.1%, 0.5%, 1%, 5%, and 10% of the composition by weight, and an upper limit chosen from 60%, 50%, 40%, 30%, 25%, 20%, and 15% of the composition by weight. In some embodiments, the inositol hexaphosphate or the salt or derivative thereof or the fucoxanthin or the derivative thereof is present in an amount of from 1% to 60% by weight of the composition.

In at least one embodiment of the present disclosure, if the composition comprises the inositol hexaphosphate or the salt or derivative thereof without the fucoxanthin or the derivative thereof, the inositol hexaphosphate or the salt or derivative thereof serves as a sole active ingredient for preventing or treating the hearing loss in the composition. In some embodiments, the inositol hexaphosphate or the salt or derivative thereof may be in combination with an additional active ingredient, e.g., other than the fucoxanthin or the derivative thereof, for preventing or treating the hearing loss.

In at least one embodiment of the present disclosure, if the composition comprises the fucoxanthin or the derivative thereof without the inositol hexaphosphate or the salt or derivative thereof, the fucoxanthin or the derivative thereof serves as a sole active ingredient for preventing or treating the hearing loss in the composition. In some embodiments, the fucoxanthin or the derivative thereof may be in combination with an additional active ingredient, e.g., other than the inositol hexaphosphate or the salt or derivative thereof, for preventing or treating the hearing loss.

In at least one embodiment of the present disclosure, the composition comprises the combination of inositol hexaphosphate or the salt or derivative thereof and fucoxanthin or the derivative thereof with or without another additional active ingredient for preventing or treating the hearing loss.

In at least one embodiment of the present disclosure, the composition provided herein may be a pharmaceutical composition or an edible composition. In some embodiments, the pharmaceutical composition of the present disclosure comprises inositol hexaphosphate or a salt or derivative thereof, fucoxanthin or a derivative thereof, or a combination thereof, and a pharmaceutically acceptable excipient thereof. In some embodiments, the edible composition of the present disclosure comprises inositol hexaphosphate or a salt or derivative thereof, fucoxanthin or a derivative thereof, or a combination thereof, and a food excipient thereof.

In at least one embodiment of the present disclosure, the pharmaceutically acceptable excipient or the food excipient is selected from the group consisting of a filler, a binder, a preservative, a disintegrating agent, a lubricant, a suspending agent, a wetting agent, a solvent, a surfactant, an acid, a flavoring agent, polyethylene glycol (PEG), alkylene glycol, sebacic acid, dimethyl sulfoxide, an alcohol, calcium stearate, microcrystalline cellulose, silicon dioxide, gelatin, fat, glycerin, dietary fiber, alginate, pectin, carrageenan, amidated pectin, xanthan, gellan gum, karaya gum, rhamsan, welan, gum ghatti, gum arabic, and any combination thereof.

In at least one embodiment of the present disclosure, the inositol hexaphosphate or the salt or derivative thereof or the fucoxanthin or the derivative thereof in the composition may be synthetic or naturally occurring. In some embodiments, the inositol hexaphosphate or the salt or derivative thereof may be naturally occurring from grains, such as soy, barley, corn, millet, rice bran, rice germ, wheat, buckwheat, fair groat, roasted groat, oak, and quinoa seed. In some embodiments, the fucoxanthin or the derivative may be naturally occurring from brown alga (e.g., Phaeophyceae), such as Undaria pinnatifida, and diatoms (e.g., Bacillariophyceae), such as Thalassiosira pseudonana.

In at least one embodiment of the present disclosure, the composition provided herein may be an edible composition comprising at least one of soy, barley, corn, millet, rice bran, rice germ, wheat, buckwheat, fair groat, roasted groat, oak, quinoa seed, seaweed, and an extract thereof.

In at least one embodiment of the present disclosure, the composition is in a form selected from the group consisting of an injection formulation, a dry powder, a tablet, an oral liquid, a flake, a film, a lozenge, a capsule, a granule, a pill, a gel, a lotion, an ointment, an emulsifier, a paste, a cream, an eye drop, and a salve.

In at least one embodiment of the present disclosure, a method for preventing or treating hearing loss in a subject in need thereof is provided. In some embodiments, the method comprises administering an effective amount of the above-mentioned composition to the subject. In some embodiments, the method comprises administering an effective amount of inositol hexaphosphate or a salt or derivative thereof to the subject. In some embodiments, the method comprises administering an effective amount of fucoxanthin or a derivative thereof to the subject.

In at least one embodiment of the present disclosure, the inositol hexaphosphate or the salt or derivative thereof and the fucoxanthin or the derivative thereof are administered simultaneously or sequentially to the subject. In case of sequential administration, the inositol hexaphosphate or the salt or derivative thereof may be administered to the subject first, followed by the administration of the fucoxanthin or the derivative thereof, and vice versa.

In at least one embodiment, the inositol hexaphosphate or the salt or derivative thereof and the fucoxanthin or the derivative thereof used in the present disclosure may be optionally formulated with one or more pharmaceutically acceptable excipients and/or one or more food excipients. In some embodiments, the inositol hexaphosphate or the salt or derivative thereof and the fucoxanthin or the derivative thereof are formulated into one single formulation that may be administered simultaneously to the subject. In some embodiments, the first agent and the second agent are each formulated into separate formulations, which may be administered simultaneously or sequentially to the subject.

In at least one embodiment of the present disclosure, the inositol hexaphosphate or the salt or derivative thereof and the fucoxanthin or the derivative thereof are independently administered to the subject in an effective amount of from about 1 mg/kg/day to about 500 mg/kg/day. In some embodiments, an effective amount of the inositol hexaphosphate or the salt or derivative thereof or the fucoxanthin or the derivative thereof administered to the subject has a lower limit chosen from 1 mg/kg/day, 3 mg/kg/day, 5 mg/kg/day, 10 mg/kg/day, 15 mg/kg/day, 20 mg/kg/day, and 30 mg/kg/day, and an upper limit chosen from 500 mg/kg/day, 400 mg/kg/day, 300 mg/kg/day, 200 mg/kg/day, 100 mg/kg/day, 90 mg/kg/day, 80 mg/kg/day, 70 mg/kg/day, 60 mg/kg/day, 50 mg/kg/day, and 40 mg/kg/day. In some embodiments, the inositol hexaphosphate or the salt or derivative thereof or the fucoxanthin or the derivative thereof is administered to the subject in an effective amount of from about 3 mg/kg/day to about 80 mg/kg/day.

In at least one embodiment of the present disclosure, the hearing loss is congenital or acquired, such as those caused by at least one of infection, noise exposure, chemotherapy, antibiotic, and aging.

In at least one embodiment of the present disclosure, the subject suffers from the hearing loss involving degeneration or loss of hair cells. In some embodiments, the hair cells may be vestibular hair cells, cochlear hair cells, or a combination thereof. In some embodiments, the subject suffers from sensorineural hearing loss, tinnitus or vertigo. In some embodiments, the subject suffers from vertigo in combination with sensorineural hearing loss. In some embodiments, the subject suffers from acute hearing loss, chronic hearing loss, primary tinnitus, Meniere's disease, or labyrinthine concussion.

In at least one embodiment of the present disclosure, the composition may be administered to the subject orally, intravenously, subcutaneously, intradermally, intrathecally, intraperitoneally, intranasally, intramuscularly, intrapleuraly, topically, or through nebulization.

In at least one embodiment of the present disclosure, a composition for use in preventing or treating hearing loss in a subject in need thereof is provided. In some embodiments, the composition comprises at least one of inositol hexaphosphate or a salt or derivative thereof and fucoxanthin or a derivative thereof, and at least one of a pharmaceutically acceptable excipient thereof and a food excipient thereof.

In at least one embodiment of the present disclosure, a use of a composition in the manufacture of a medicament for preventing or treating hearing loss in a subject in need thereof is provided. In some embodiments, the composition comprises at least one of inositol hexaphosphate or a salt or derivative thereof and fucoxanthin or a derivative thereof, and at least one of a pharmaceutically acceptable excipient thereof and a food excipient thereof.

In some embodiments, the inositol hexaphosphate or the salt or derivative thereof, the fucoxanthin or the derivative thereof, and the combination thereof provided in the present disclosure may protect the hair cells and improve the hearing thresholds, and thus can be useful for preventing or treating the hearing loss, such as that caused by infection, noise exposure, chemotherapy, antibiotic, and aging, as well as improving the quality of life of patients.

### BRIEF DESCRIPTION OF THE DRAWINGS

The present disclosure can be more fully understood by reading the following detailed descriptions of the embodiments, with reference made to the accompanying drawings.

FIGS. 1A to 1C show the experimental design for three animal models of hearing loss. FIG. 1A shows that for the cisplatin-induced SNHL in mice, IP6 (40 mg/kg or 80 mg/kg) was injected intraperitoneally (IP) into 2-month-old mice, and two hours after IP6 administration, 3 mg/kg cisplatin was injected intraperitoneally for 5 consecutive days to induce SNHL. One week later, the hearing threshold of mice was measured using an auditory brain response (ABR) instrument. Afterwards, the mice were euthanized, and their cochlear tissues were collected for immunohistochemical staining. FIG. 1B shows that for the kanamycin/furosemide-induced SNHL in mice, kanamycin (1 mg/g) and furosemide (0.1 mg/g) were injected subcutaneously (SC) or intraperitoneally (IP) on the first day, and IP6 (80 mg/kg), fucoxanthin (60 mg/kg), or their combination (80 mg/kg of IP6 and 30 mg/kg of fucoxanthin) was injected intraperitoneally for 5 consecutive days. One week later, the ABR hearing threshold was measured, and the mice's cochlear tissues were collected. FIG. 1C shows that for mice with age-elated hearing loss, IP6 (40 mg/kg or 80 mg/kg) was injected intraperitoneally for 3 days/week for 10 months or received with 2% through drinking water for 10 months. The ABR hearing threshold was measured every three months. When the experimental mice were 12 months of age, the mice were euthanized, and their cochlear tissues were collected.

FIGS. 2A to 2D show the effects of IP6 injection on the loss of hair cells in cochlea tissues from cisplatin-injected mice. FIGS. 2A to 2C are the representative results of immunohistochemical staining of the nucleus (stained by DAPI, blue), hair cells (HCs, stained by Myo7A, red), and cytoskeleton (stained by phalloidin, green) in the apex (FIG. 2A), middle (FIG. 2B) and base (FIG. 2C) regions of the cochlear tissues in each group of mice. Scale bar=25 μm. Arrows indicate the loss of outer hair cells (OHCs) in the cochlear tissues. FIG. 2D shows the quantification results of the loss of hair cells in FIGS. 2A to 2C. Cis: mice treated with cisplatin (3 mg/kg/day, IP). Cis+IP6: mice treated with cisplatin (3 mg/kg/day, IP) and IP6 (80 mg/kg/day, IP).

FIGS. 3A and 3B show the effects of IP6 injection (40 mg/kg or 80 mg/kg) on hearing threshold of cisplatin-injected mice. FIG. 3A shows the representative results of auditory brain response (ABR) at the frequency of 12 kHz in each group, and FIG. 3B shows the average hearing threshold of each group. Cis: mice treated with cisplatin (3 mg/kg/day, IP). Cis+IP6 40: mice treated with cisplatin (3 mg/kg/day, IP) and IP6 (40 mg/kg/day, IP). Cis+IP6 80: mice treated with cisplatin (3 mg/kg/day, IP) and IP6 (80 mg/kg/day, IP).

FIGS. 4A to 4D show the effects of IP6, fucoxanthin, or their combination on hearing threshold of kanamycin (Kana)/furosemide (Furo)-injected mice. FIG. 4A shows the representative results of auditory brain response (ABR) at the frequency of 12 kHz in each group. FIGS. 4B and 4C show the average hearing threshold of each group. FIG. 4D shows the representative results of immunohistochemical staining of the nucleus (stained by DAPI, blue), hair cells (stained by Myo7A, red), and cytoskeleton (stained by phalloidin, green) in the apex, middle and base regions of the cochlear tissues in each group of mice. Scale bar=25 μm. White boxes indicate the loss of outer hair cells (OHCs) in the cochlear tissues. Kana+Furo: mice treated with kanamycin and furosemide. Kana+Furo+IP6: mice treated with kanamycin, furosemide, and IP6 (80 mg/kg/day, IP). Kana+Furo+IP6 80+Fuco 30: mice treated with kanamycin, furosemide, IP6 (80 mg/kg/day, IP), and fucoxanthin (30 mg/kg/day, IP). Kana+Furo+Fuco 60: mice treated with kanamycin, furosemide, and fucoxanthin (60 mg/kg/day, IP).

FIGS. 5A to 5E show the neuropathological gene panel analyses of protective effects of IP6 against kanamycin (Kana)/furosemide (Furo)-induced hearing loss in mice. FIG. 5A shows the gene scores of the biological pathways in the mRNA expression profiles of the cochlear tissues of each group. FIG. 5B shows the significantly changed expression of genes in the gene scores for microglia, endothelial cells, and neurons in the cochlear tissues of each group. FIG. 5C shows the heat map of the gene expression profiles for each biological pathway after normalization on the basis of the control group. FIGS. 5D and 5E show the significantly changed expression of genes in the gene scores for vesicle trafficking, myelination, lipid metabolism, and carbohydrate metabolism in the cochlear tissues of each group. Kana+Furo: mice treated with kanamycin and furosemide. Kana+Furo+IP6: mice treated with kanamycin, furosemide, and IP6 (80 mg/kg/day, IP).

FIGS. 6A to 6G show the effects of IP6 injection (40 mg/kg or 80 mg/kg) or IP6 drinkable solution on hearing thresholds of aging mice (6-, 9- and 12-month-old). FIG. 6A to 6E show the average hearing threshold of each group measured by auditory brain response (ABR) at the frequency of 12 kHz, and FIG. 6F shows the representative results of ABR at the frequency of 12 kHz in each group when the mice are 12-month-old. FIG. 6G shows the representative results of immunohistochemical staining of the nucleus (stained by DAPI, blue), hair cells (stained by Myo7A, red), and cytoskeleton (stained by phalloidin, green) in the apex, middle and base regions of the cochlear tissues in each group of mice. Scale bar=25 μm. Arrows indicate the loss of inner hair cells (IHCs) in the cochlear tissues. White boxes indicate the loss of outer hair cells (OHCs) in the cochlear tissues. IP6 40: mice treated with IP6 (40 mg/kg/day, IP). IP6 80: mice treated with IP6 (80 mg/kg/day, IP). IP6 drink: mice treated with IP6 (2%, orally).

### DETAILED DESCRIPTION OF THE EMBODIMENTS

The description discloses some embodiments in such a detail that a person skilled in the art is able to utilize the embodiments based on the disclosure. Not all steps or features of the embodiments are discussed in detail, as many of the steps or features will be obvious to the person skilled in the art based on this disclosure.

It is further noted that, as used in this disclosure, the singular forms “a,” “an,” and “the” include plural referents unless expressly and unequivocally limited to one referent. The term “or” is used interchangeably with the term “and/or” unless the context clearly indicates otherwise.

As used herein, the term “about” generally means within 10%, 5%, 1%, or 0.5% of a given value or range. Alternatively, the term “about” means within an acceptable standard error of the mean when considered by one of ordinary skill in the art. Unless otherwise expressly specified, all of the numerical ranges, amounts, values and percentages such as those for quantities of materials, durations of time periods, temperatures, operating conditions, ratios of amounts, and the likes disclosed herein should be understood as modified in all instances by the term “about.”

The present disclosure provides a method of preventing or treating hearing loss, such as chronic or acute hearing loss caused by chemotherapy or antibiotic, in a subject in need thereof. In some embodiments of the present disclosure, the method comprises administering an effective amount of a composition to the subject, wherein the composition comprises at least one of inositol hexaphosphate or a salt or derivative thereof and fucoxanthin or a derivative thereof, and at least one of a pharmaceutically acceptable excipient thereof and a food excipient thereof.

As used herein, the term “comprising” or “comprises” is used in reference to compositions, methods, and respective component(s) thereof, which are included in the present disclosure, yet open to the inclusion of unspecified elements.

As used herein, the term “treat,” “treating” or “treatment” encompasses partially or completely preventing, ameliorating, mitigating and/or managing a symptom, a disorder or a condition associated with a disease. The term “treat,” “treating” or “treatment” as used herein refers to application or administration of one or more therapeutic agent or surgery to a subject, who has a symptom, a disorder or a condition associated with a disease, with the purpose to partially or completely alleviate, ameliorate, relieve, delay onset of, inhibit progression of, reduce severity of, and/or reduce incidence of one or more symptoms, disorders or conditions associated with the disease. Treatment may be administered to a subject who exhibits only an early sign of such symptoms, disorders, and/or conditions for the purpose of decreasing the risk of developing the symptoms, disorders, and/or conditions associated with a disease.

As used herein, the term “preventing” or “prevention” refers to preventive or avoidance measures for a disease or symptoms or conditions of a disease, which include, but are not limited to, applying or administering one or more active agents to a subject who has not yet been diagnosed as a patient suffering from the disease or the symptoms or conditions of the disease but may be susceptible or prone to the disease. The purpose of the preventive measures is to avoid, prevent, or postpone the occurrence of the disease or the symptoms or conditions of the disease.

As used herein, the terms “patient,” “individual” and “subject” are used interchangeably. The term “subject” means a human or animal. Examples of the subject include, but are not limited to, a rodent, a murine, a monkey, a guinea pig, a dog, a cat, a cow, a sheep, a pig, a horse, a rabbit, and a human. In some embodiments of the present disclosure, the subject is a mammal, e.g., a primate such as a human.

As used herein, the phrase “an effective amount” refers to the amount of an active ingredient (e.g., inositol hexaphosphate, fucoxanthin or a combination thereof) that is required to confer a desired effect (e.g., protecting hair cells) on the treated subject. Effective doses will vary, as recognized by one of ordinary skill in the art, depending on routes of administration, excipient usage, the possibility of co-usage with other treatment, and the condition to be treated.

As used herein, the term “administering” or “administration” refers to the placement of an active ingredient (e.g., inositol hexaphosphate, fucoxanthin or a combination thereof) into a subject by a method or route which results in at least partial localization of the active ingredient at a desired site to produce a desired effect. The active ingredient described herein may be administered by any appropriate route known in the art including, but not limited to, oral or parenteral routes, including intraperitoneal, intravenous, intradermal, intramuscular, subcutaneous, or transdermal routes.

In at least one embodiment of the present disclosure, the composition may be administered to the subject 1 to 4 times per day, 1 to 7 times per week, or 1 to 10 times per month. In some embodiments, the composition is administered to the subject with a frequency selected from the group consisting of once per month, 2 times per month, 3 times per month, 4 times per month, 5 times per month, 6 times per month, 7 times per month, 8 times per month, once per week, 2 times per week, 3 times per week, 4 times per week, 5 times per week, 6 times per week, once every three days, 2 times every three days, 3 times every three days, once every two days, 2 times every two days, once per day, and 2 times per day.

The composition of the present disclosure may comprise the inositol hexaphosphate or the salt or derivative thereof as a sole active ingredient for preventing or treating hearing loss. In other words, the inositol hexaphosphate or the salt or derivative thereof serves as the only active ingredient for the auditory dysfunction in the composition. Alternatively, the composition of the present disclosure may comprise the fucoxanthin or the derivative thereof as a sole active ingredient for preventing or treating hearing loss. In other words, the fucoxanthin or the derivative thereof serves as the only active ingredient for the auditory dysfunction in the composition. In this embodiment, the present disclosure provides a safe and effective therapy for preventing or treating hearing loss by the use of the inositol hexaphosphate or the salt or derivative thereof or the fucoxanthin or the derivative thereof alone as the active ingredient.

Alternatively, in other embodiments, the composition may comprise a combination of the inositol hexaphosphate or the salt or derivative thereof and the fucoxanthin or the derivative thereof. In this embodiment, the combination has a synergistic effect in rescuing loss of cochlear hair cells and reducing hearing thresholds, thereby safely and effectively preventing or treating hearing loss.

As used herein, the term “synergistic” refers to a combination of therapies which is more effective than those of single therapies.

Alternatively, in other embodiments, in addition to the inositol hexaphosphate or the salt or derivative thereof and the fucoxanthin or the derivative thereof, the composition may be administered to a subject in combination with another active ingredient for hearing loss unless the effect of the present disclosure is inhibited. In these embodiments, the inositol hexaphosphate or the salt or derivative thereof and/or the fucoxanthin or the derivative thereof may be provided with the another active ingredient in a single formulation or in separate formulations.

The composition of the present disclosure may be a pharmaceutical composition comprising a pharmaceutically acceptable excipient or an edible composition comprising a food excipient.

As used herein, the term “pharmaceutically acceptable excipient” refers to a pharmaceutically acceptable material, composition, or vehicle, such as a disintegrating agent, a binder, a lubricant, and a surfactant, which does not abrogate the biological activity or properties of the active ingredient, and is relatively non-toxic; that is, the material may be administered to an individual without causing an undesirable biological effect or interacting in a deleterious manner with any of the components of the composition in which it is contained.

As used herein, the term “food excipient” refers to an edible material that may have no bioactivity itself but improve the absorption of nutrients and active ingredients into the body; for example, the food excipient may protect nutrients and ingredients until they arrive at the location where they are intended to be useful.

In at least one embodiment of the present disclosure, the edible composition provided herein may comprise a grain enriched with inositol hexaphosphate or a salt or derivative thereof. The examples of the grain enriched with inositol hexaphosphate or the salt or derivative thereof include, but are not limited to, rice bran, rice germ, and buckwheat. For example, it has been reported that the content of inositol hexaphosphate in rice bran may range from 0.22% to 2.22%^{[1]}. Based on the analysis method disclosed by Lehrfeld^{[2]}, it was found that 100 g of a rice germ extract may contain inositol hexaphosphate in an amount of about 509 mg.

In at least one embodiment of the present disclosure, the edible composition provided herein may comprise seaweed or an extract thereof enriched with fucoxanthin or a derivative thereof.

Many examples have been used to illustrate the present disclosure. The examples below should not be taken as a limit to the scope of the disclosure.

### EXAMPLES

### Materials and Methods

The materials and methods used in the following Examples 1 to 3 are described in detail below. The materials used in the present disclosure but unannotated herein are commercially available.

### (1) Animal Models

Animal Care Committee of Mackay Medical College approved the animal experiments. C57BL/6J female mice (8-weeks-old) were purchased from BioLasco (Taiwan). All animals were bred and housed following animal protocols.

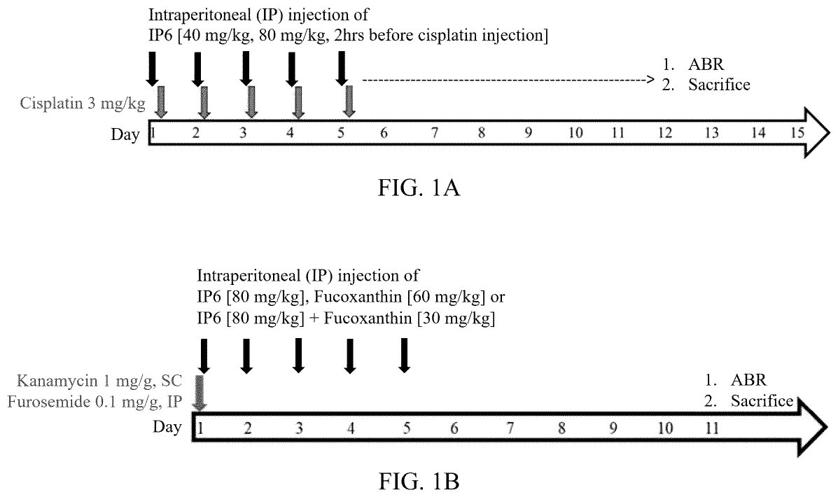

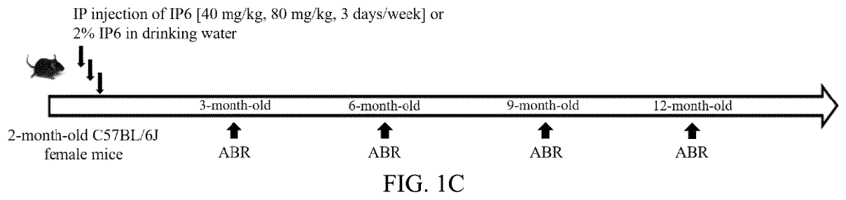

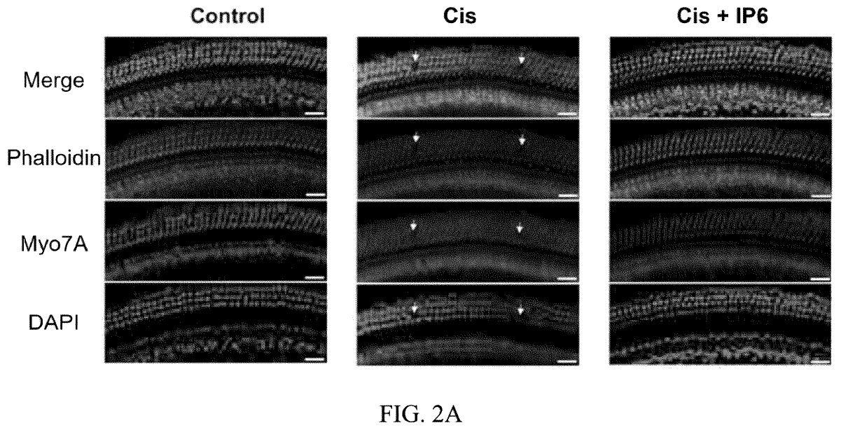

For the cisplatin-induced sensorineural hearing loss (SNHL) in mice, the schematic protocol for the dosing and timing of cisplatin and IP6 was presented in FIG. 1A. Specifically, cisplatin was dissolved in 0.1% dimethyl sulfoxide (DMSO) to induce ototoxicity in mice through intraperitoneal (IP) injection. The used procedures followed the protocol reported by Wu et al.^{[3]}. Inositol hexaphosphate (IP6) obtained from Sigma (USA) was dissolved in ddH₂O, and the doses given to the experimental mice intraperitoneally were 40 mg/kg and 80 mg/kg (100 μL of IP6). The mice were first treated with IP6 through intraperitoneal injection 2 hours before being injected with cisplatin; subsequently, the mice underwent IP6 treatment for 4 consecutive days. The mice of the control group received only phosphate-buffered saline (PBS, 100 μL). For the serum biochemical markers, i.e., aspartate aminotransferase (AST), alanine aminotransferase (ALT), and total bilirubin, there is no difference between the control mice, the mice injected with cisplatin, and the mice injected with cisplatin and IP6 (data not shown).

For the kanamycin-induced SNHL in mice, the schematic protocol for the dosing and timing of kanamycin, furosemide, IP6 and/or fucoxanthin was presented in FIG. 1B. Kanamycin (Sigma Aldrich, USA) was dissolved in PBS, and kanamycin (1 mg/g) and furosemide (0.1 mg/g, Sigma Aldrich, USA) were injected as a single dose on the first day. Specifically, furosemide was intraperitoneally injected within 30 min after kanamycin was injected subcutaneously (SC). One hour later, IP6 and/or fucoxanthin was intraperitoneally injected for 5 consecutive days.

For the mouse model of age-related hearing loss (ARHL), female C57BL/6J mice at the following ages were used in the present study^{[4]}. The schematic protocol for the ARHL mouse model was presented in FIG. 1C. For acclimatization, the animals were housed for at least 1 week before the initiation of the experiments. The animals were maintained under a 12-h light/dark cycle at 20° C. to 26° C. and 40% to 70% humidity, and were allowed free access to normal food and water until experimentation. IP6 was dissolved in ddH₂O, and the doses given to the experimental mice intraperitoneally were 40 mg/kg and 80 mg/kg. Additionally, IP6 was dissolved in ddH₂O to make an IP6 solution (2%) and given to the mice in drinking water.

All mice were under anesthesia when their auditory brainstem response (ABR) was measured. Cochlear tissues were collected for further immunohistochemical and gene expression analyses.

### (2) Whole-Mount Dissection, Confocal Microscopy, and the Loss of Outer Hair Cells (OHCs) Count

Experimental mice were anesthetized, and their cochlear tissues were isolated and dissected. For whole-mount dissection, the protocol detailed by Neal et al. was followed^{[5]}. In brief, the cochlea was decalcified for 1 hour using a decalcification solution purchased from Apex Engineering (Aurora, IL, USA). The excess temporal bone around the cochlea was trimmed, and the cochlea was bisected along the midmodiolar plane and then submerged in phosphate buffered saline (PBS). A series of cuts through the modiolus separated the half-turns from one another. Each half-turn was then removed from the surrounding temporal bone, and the tectorial membrane was removed. The cochlear tissues were fixed with 4% paraformaldehyde. The tissues were incubated with the primary antibodies of anti-Myo7A (1:100, Santa Cruz, CA) and anti-phalloidin conjugated with Alexa 488 (1:200, Thermo Fisher Scientific) at 4° C. overnight and at 4° C. for 2 hours, respectively. The tissues were incubated with the secondary antibody of rhodamine goat anti-mouse (1:200, KPL) at room temperature for 2 hours. Cells were stained with 4′,6-diamidino-2-phenylindole (DAPI) (Sigma).

For the loss of OHCs counts, hair cells (HCs) were counterstained with phalloidin, Myo7A and DAPI stainings. Images were captured using a confocal spectral microscope (Leica SP8). The images at three randomly-selected regions (300 μm length) in the apical, middle, and basal cochlear turns were acquired by using the same apparatus, and the loss of OHCs was manually counted. Average loss of OHCs was then calculated in apex, middle and base regions, respectively^{[6]}. Fluorescent images were taken with a Leica SP8 confocal fluorescence microscope.

### (3) Auditory Brainstem Response (ABR) Measurement

The ABR of mice was measured to evaluate their hearing threshold. In brief, measurements were performed on the scalps of mice under anesthesia by using subdermal needle electrodes placed at the vertex, below the pinna of the left ear (as a reference measurement), and below the contralateral ear (ground). The sound stimuli included clicks (100-ms duration; 4, 8, and 12 kHz). The devices used for ABR measurement were specifically designed for animal experiments (BIOPAC System, USA). During the measurement of hearing threshold, acoustic stimuli were applied directly to the ear canal at a stimulus intensity ranging from 100-dB to a threshold 10-dB sound pressure level at 10-dB intervals near the threshold level. The ABR threshold was measured through detection of the presence of V waves. All procedures for ABR measurement were modified from previous reports^{[7-11]}.

### (4) NanoString Gene Expression Analysis

Total mRNA was extracted from mouse cochlear tissues as per the protocol described in previous studies^{[10, 11]}. The expression profile of neuropathological genes was analyzed using a NanoString mouse neuropathology panel (NanoString Technologies, Seattle, WA, USA), which includes genes involved in neural cell types and neuropathological processes^{[12, 13]}. All analyses of genes or pathways scores were performed in accordance with the user manual of the nCounter Advanced Analysis Software and were described in detail in previous studies^{[10, 11]}. Cold Spring Biotech in Taiwan was responsible for performing the NanoString gene expression analysis and bioinformatics analyses.

### (5) Statistical Analyses

Data were expressed as means±standard deviation (SD). One-way analysis of variance was used to perform multigroup comparison. Statistical significance was defined as P<0.05.

### Example 1: Effect of IP6 on the Chronic Cisplatin-Induced OHC Loss in Mice

In this example, 8-week-old mice were randomly divided into three groups (6 to 9 mice per group), in which those of the control group received 100 μL of PBS once per day for 5 consecutive days; those of the cisplatin (Cis) group received intraperitoneal injection of cisplatin (3 mg/kg) once per day for 5 consecutive days; and those of the Cis+IP6 group were injected with IP6 (80 mg/kg), followed by cisplatin (3 mg/kg) 2 hours later, once per day for 5 consecutive days. One week later, the mice were sacrificed to obtain cochlear tissues for immunohistochemical staining and OHCs count.

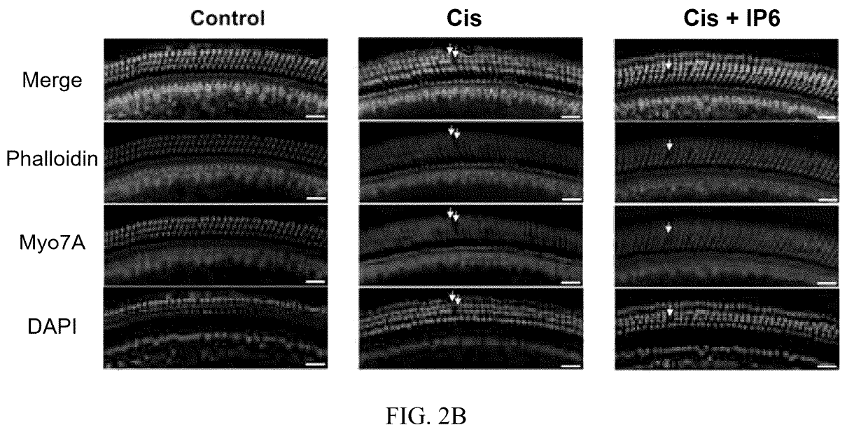

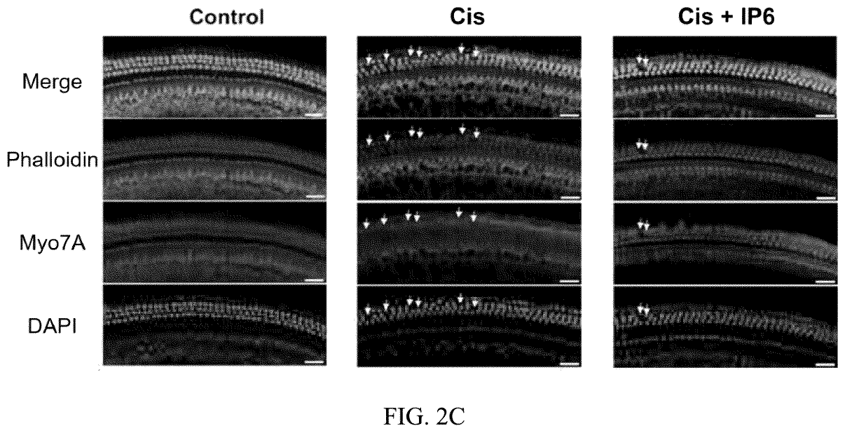

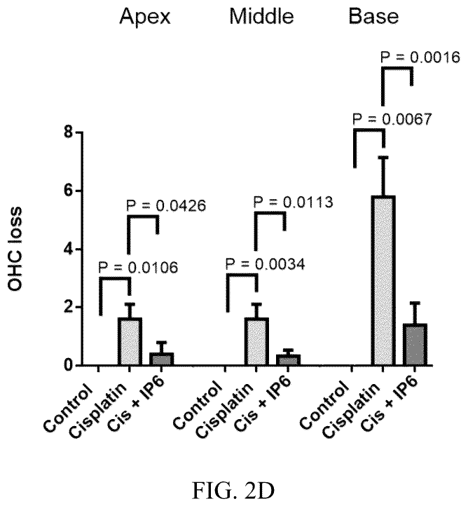

As shown in FIGS. 2A to 2D, the results for the OHCs (Myo7A⁺) loss of cochlear tissues showed that the cisplatin-injected mice had a significant loss of OHCs in the apex, middle, and base regions compared with the control group. After IP6 treatment in cisplatin-injected mice, the loss of OHCs in the apex, middle and base regions was significantly reduced compared with that of the cisplatin-injected mice that received no IP6 treatment. These results indicated that IP6 may effectively protect against OHC loss induced by cisplatin.

### Example 2: Effect of IP6 on the Chronic Cisplatin-Induced Hearing Loss in Mice

In this example, 8-week-old female mice were randomly divided into four groups (6 to 9 mice per group), in which those of the control group received 100 μL of PBS once per day for 5 consecutive days; those of the Cis group received intraperitoneal injection of cisplatin (3 mg/kg) once per day for 5 consecutive days; those of the Cis+IP6 40 group were injected with IP6 (40 mg/kg), followed by cisplatin (3 mg/kg) 2 hours later, once per day for 5 consecutive days; and those of the Cis+IP6 80 group were injected with IP6 (80 mg/kg), followed by cisplatin (3 mg/kg) 2 hours later, once per day for 5 consecutive days. One week later, the hearing threshold of all groups of mice was measured using an auditory brain response (ABR) instrument.

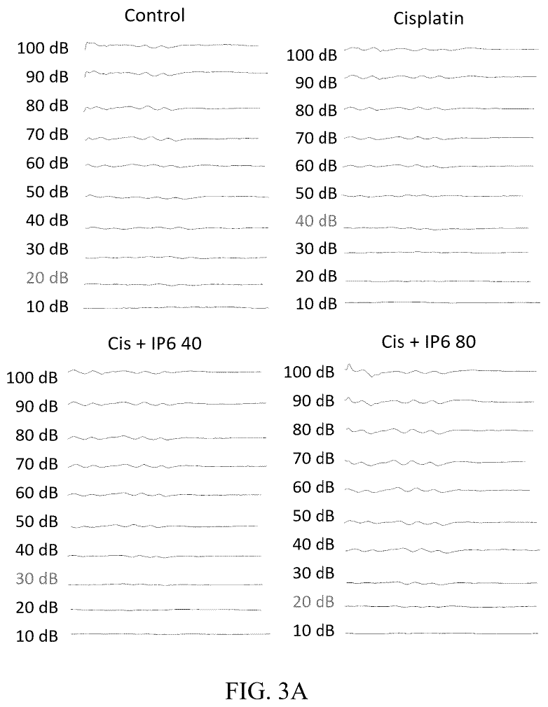

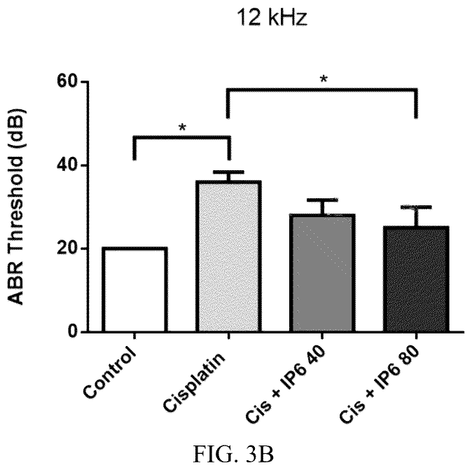

The results were shown in FIGS. 3A and 3B, indicating that the hearing threshold of the cisplatin-injected mice was significantly higher than that of the control mice, and after IP6 treatment, the hearing threshold of the cisplatin-injected mice was significantly decreased from 40 decibel (db) to about 20 to 30 db. It thus can be found that IP6 may effectively improve chronic hearing loss induced by cisplatin.

### Example 3: Effect of IP6 and/or Fucoxanthin (Fuco) on the Kanamycin (Kana)/Furosemide (Furo)-Induced Hearing Loss in Mice

In this example, 8-week-old female mice were randomly divided into five groups (6 to 9 mice per group), in which (1) those of the control group received 100 μL of PBS once per day for 5 consecutive days; (2) those of the kanamycin/furosemide group received injection of 1 mg/g dose for kanamycin and 0.1 mg/g dose for furosemide; (3) those of the Kana+Furo+IP6 group were injected with IP6 (80 mg/kg) 1 hour after the kanamycin/furosemide injection on the first day and the remaining four days; (4) those of the Kana+Furo+IP6 80+Fuco 30 group were injected with IP6 (80 mg/kg) and fucoxanthin (30 mg/kg) 1 hour after the kanamycin/furosemide injection on the first day and the remaining four days; and (5) those of the Kana+Furo+Fuco 60 group were injected with fucoxanthin (60 mg/kg) 1 hour after the kanamycin/furosemide injection on the first day and the remaining four days. One week later, the hearing threshold of all groups of mice was measured using ABR instrument. After the ABR measurement, the mice were sacrificed to obtain cochlear tissues for immunohistochemical staining.

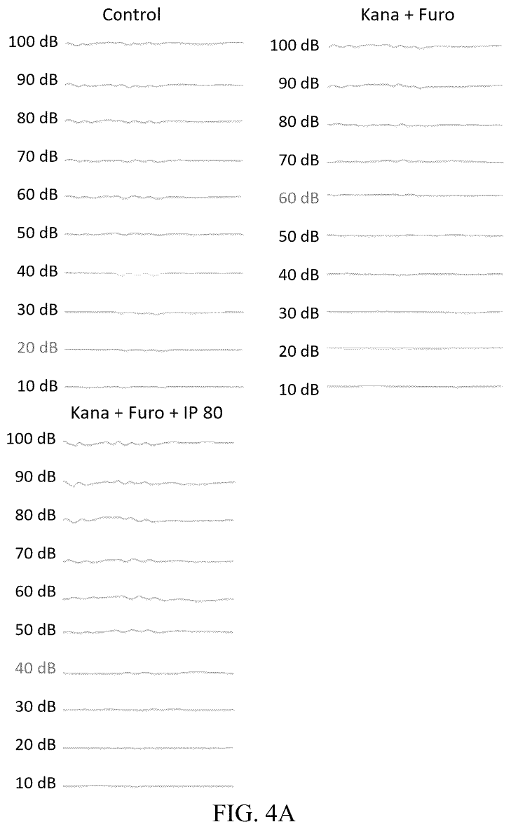

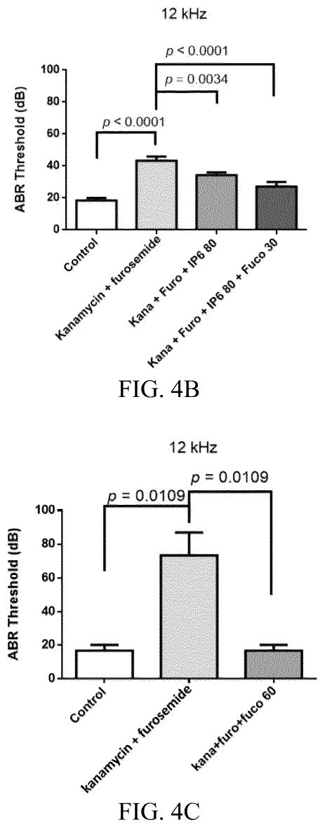

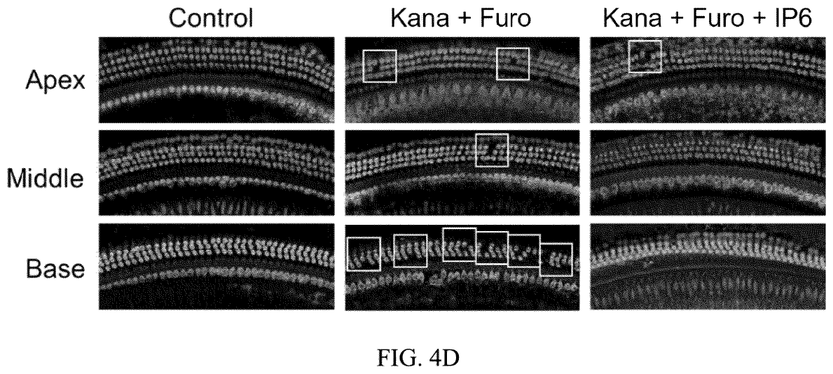

The results were shown in FIGS. 4A to 4C, indicating that the kanamycin/furosemide-injected mice had a significantly higher hearing threshold relative to the control mice, and IP6 treatment (80 mg/kg) or fucoxanthin treatment (60 mg/kg) for 5 consecutive days resulted in a significant reduction in their hearing threshold. Further, the combined administration of IP6 and fucoxanthin further revealed a synergistic effect in reducing the hearing threshold.

Additionally, FIG. 4D showed the results for the OHCs (Myo7A⁺) loss of cochlear tissues and indicated a significant loss of OHCs in the apex, middle, and base regions of the cochlear tissues of kanamycin/furosemide-injected mice. Among the kanamycin/furosemide-injected mice, 80 mg/kg IP6 treatment significantly reduced the loss of OHCs in the apex, middle, and base regions of the cochlear tissues. These results indicated that IP6 may effectively protect against OHC loss and improve hearing loss induced by kanamycin and furosemide.

### Example 4: Neuropathological Gene Expression Analysis Revealed Dysregulation in Mouse Cochlear Tissues after Kanamycin/Furosemide and IP6 Treatments

For the analyses of NanoString gene expression profiles, a NanoString neuropathology gene expression panel was used to analyze the relative RNA abundance of the three representative cochlear samples from mice of the groups as described in Example 3, i.e., the control group (n=3, hearing thresholds of 10, 20, and 20 dB at 12 kHz for all three samples), the kanamycin/furosemide group (n=3, hearing thresholds of 50, 50, and 60 dB at 12 kHz for all three samples), and the Kana+Furo+IP6 group (n=3, hearing threshold of 30, 40 and 40 dB at 12 kHz for all three samples).

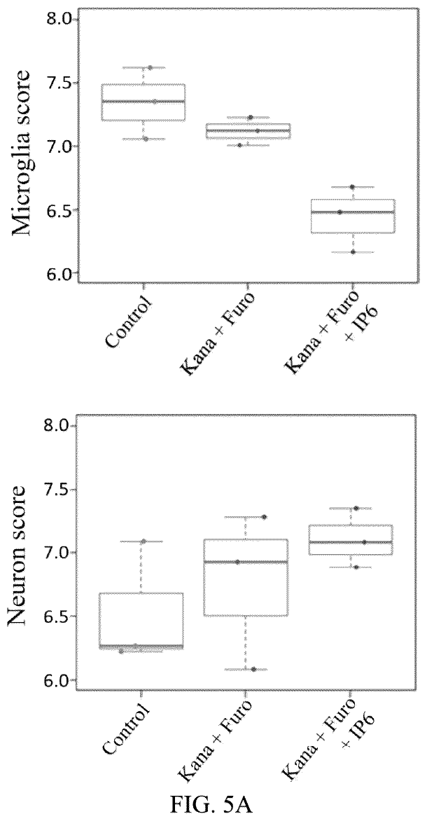

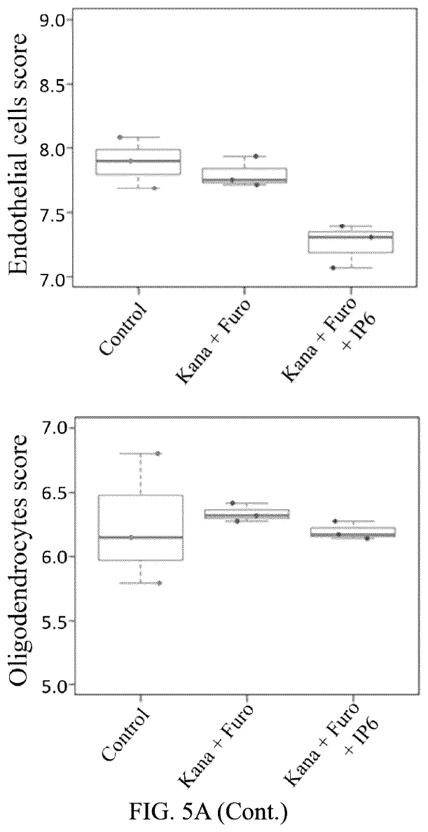

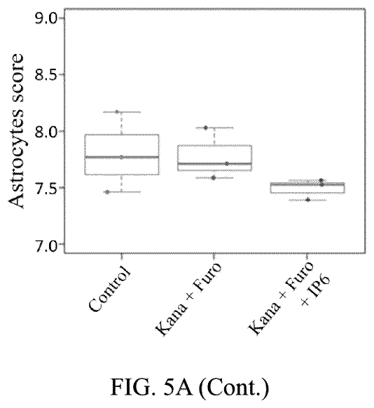

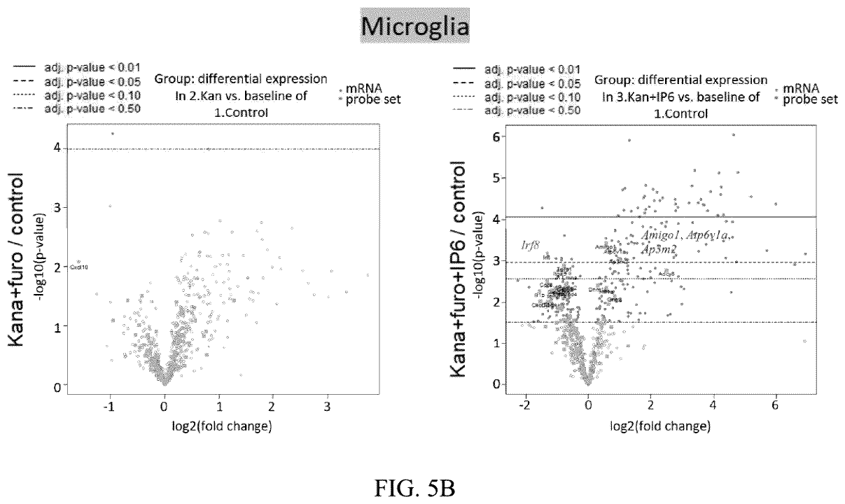

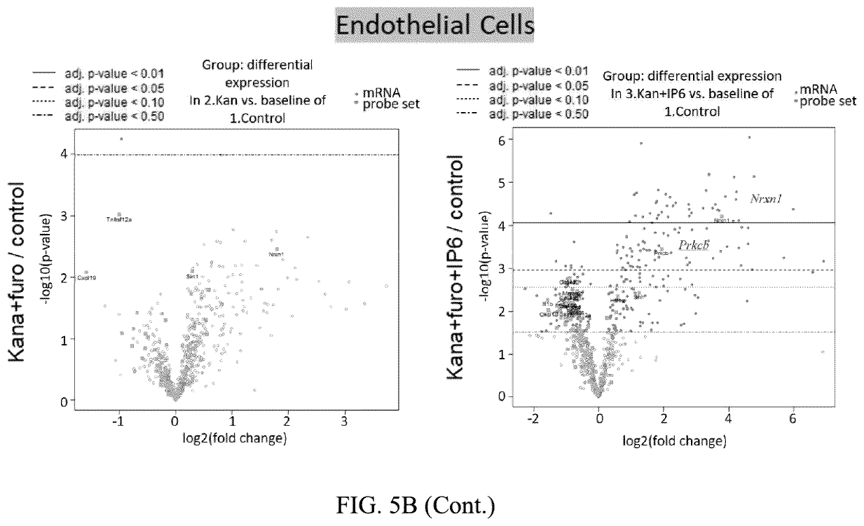

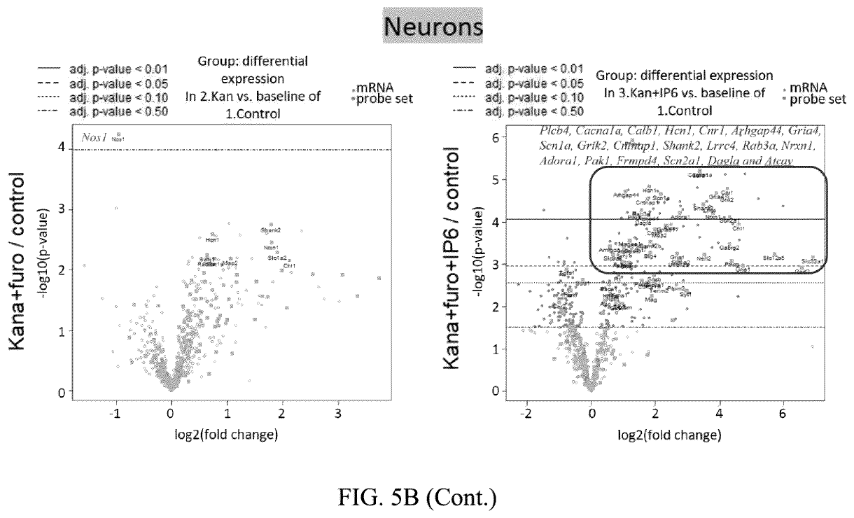

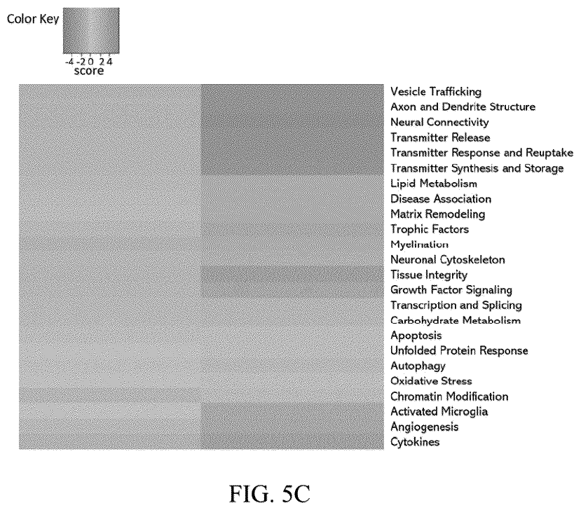

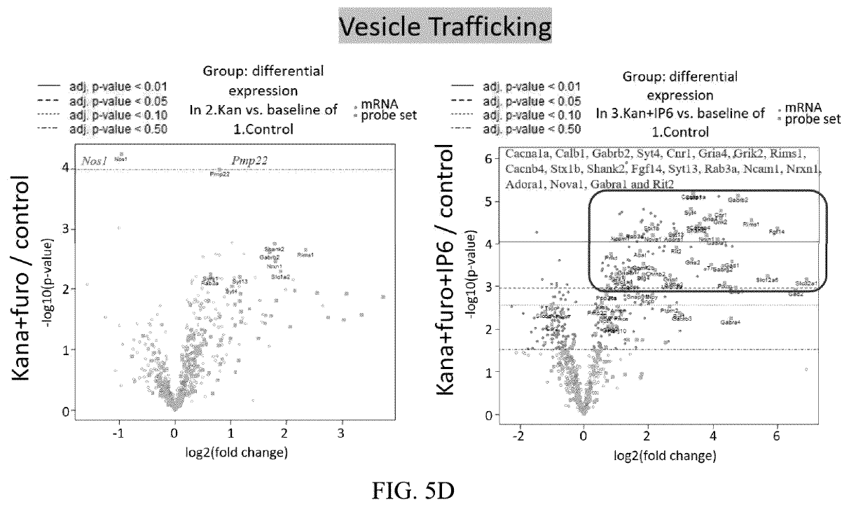

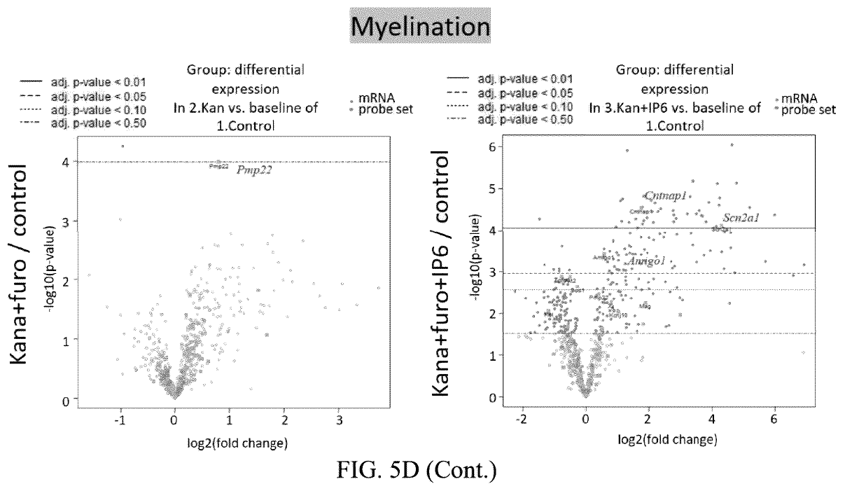

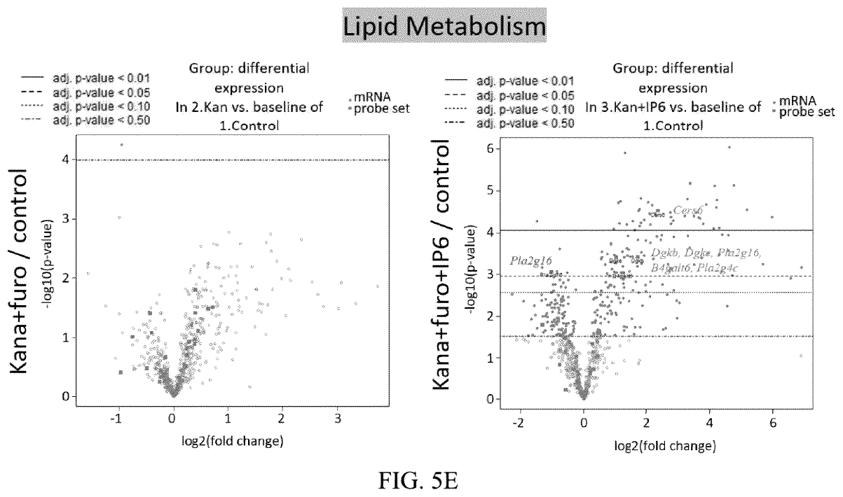

As shown in FIG. 5A, in the kanamycin/furosemide-injected mice, the transcriptional patterns of astrocytes, endothelial cells, microglia, and neurons could be reversed through 80 mg/kg IP6 treatment. Further, FIG. 5B showed that after IP6 treatment, the microglia gene score indicated that the mRNA levels of Amigo1, Atp6v1a, and Ap3m2 were significantly upregulated (P<0.05). In addition, the endothelial cell gene score indicated that the mRNA levels of Nrxn1 (P<0.01) and Prkcb (P<0.05) were significantly upregulated. IP6 treatment also significantly upregulated numerous neuron score genes, such as Plcb4, Cacna1a, Calb1, Hen1, Cur1, Arhgap44, Gria4, Scn1a, Grik2, Cntnap1, Shank2, Lrrc4, Rab3a, Nrxn1, Adora1, Pak1, Frmpd4, Scn2a1, Dagla, and Atcay (P<0.01).

Compared with the gene scores for the cochlear tissues of the kanamycin/furosemide-injected mice, the directed global significance scores suggested that IP6 treatment upregulated numerous neural repair processes, including vesicle trafficking, axon and dendrite structure formation, neural connectivity, neural transmitter release, neural transmitter response and reuptake, neural transmitter synthesis and storage, growth factor signaling, myelination, carbohydrate metabolism, and lipid metabolism (FIG. 5C).

After IP6 treatment, the gene scores pertaining to vesicle trafficking indicated that the mRNA levels of Cacna1a, Calb1, Gabrb2, Syt4, Cnr1, Gria4, Grik2, Rims1, Cacnb4, Stx1b, Shank2, Fgf14, Syt13, Rab3a, Ncam1, Nrxn1, Adora1, Nova1, Gabra1, and Rit2 were significantly upregulated (P<0.01). In addition, the myelination gene score indicated that the mRNA levels of Cntnap1 (P<0.01), Scn2a1, and Amigo1 (P<0.05) were significantly upregulated. IP6 treatment also upregulated the mRNA levels of Cers6, Dgkb, Dgke, B4galt6, and Pla2g4c, which were involved in lipid metabolism (P<0.05), and the mRNA levels of Akt3, Slc1a1, and Slc1a2, which were involved in carbohydrate metabolism (FIGS. 5D and 5E).

### Example 5: Effect of IP6 Injection on Hearing Threshold of Aging Mice

In this example, 2-month-old female mice were randomly divided into four groups (6 to 9 mice per group), in which those of the IP6 40 group received intraperitoneal injection of 40 mg/kg IP6 three days per week for 10 months; those of the IP6 80 group received intraperitoneal injection of 80 mg/kg IP6 three days per week for 10 months; those of the IP6 drink group received IP6 by adding 2% IP6 to their drinking water for 10 months (the total intake amount of IP6 was about 15 g to 20 g per mice); and those of the control group received intraperitoneal injection of PBS three days per week for 10 months. The ABR measurement was performed when the mice were 6-, 9-, and 12-month-old.

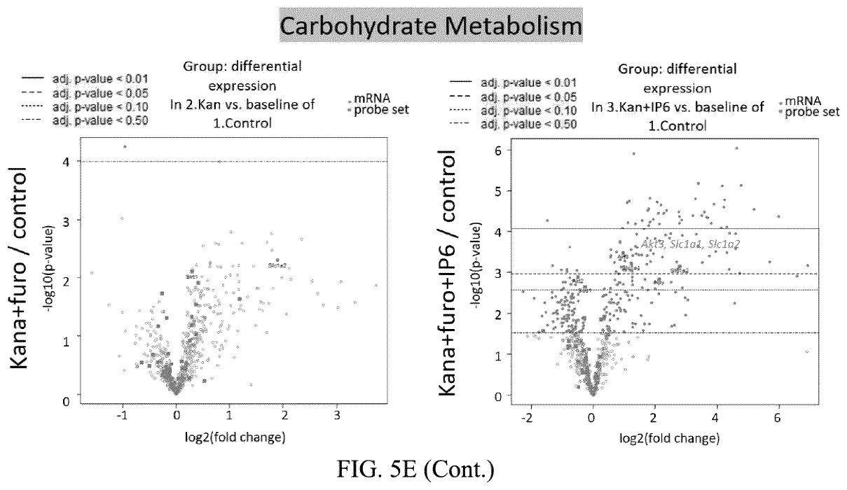

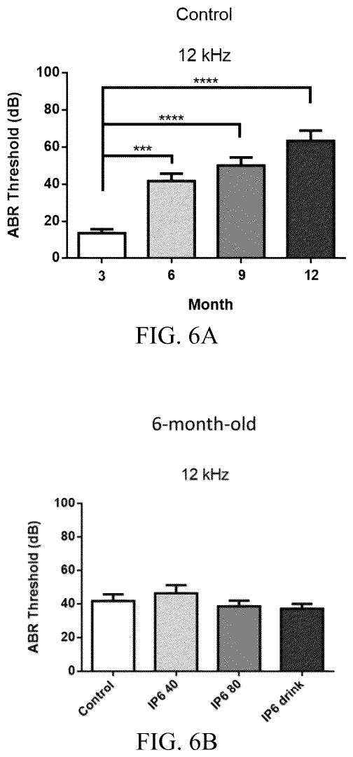

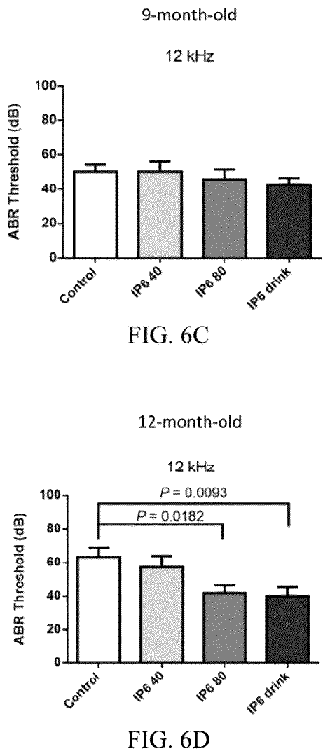

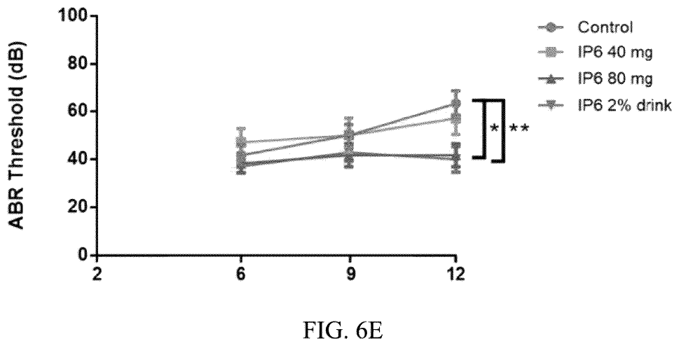

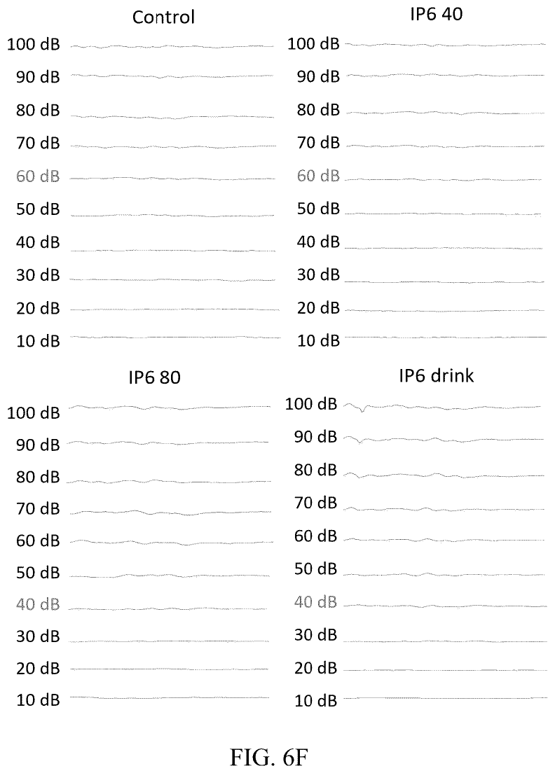

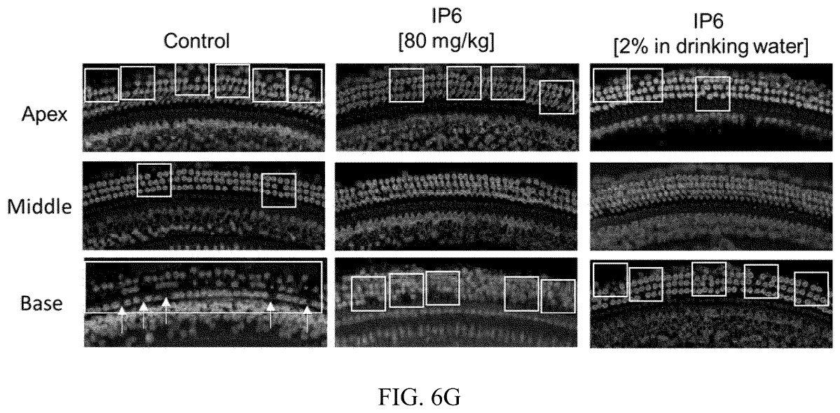

FIG. 6A showed the increase of the hearing thresholds of aging mice, while the results of ABR measurements as shown in FIGS. 6B to 6F indicated that the mice of the IP6 drink group had significantly improved hearing function at the frequency of 12 kHz compared with the control group at 12-month-old age. The hearing threshold of the mice of the IP6 drink group was reduced by 30% (approximately 20 dB) compared with that of the mice of the control group that did not receive IP6 treatment.

Also, as shown in FIG. 6G, in the 12-month-old C57BL/6J mice, the administration of IP6 (80 mg/kg) or 2% IP6 through drinking water for 10 months moderately mitigated the loss of OHCs (Myo7A⁺) in the apex, middle, and base regions of the mice's cochlear tissues. In the base region of the cochlear tissues of 12-month-old C57BL/6J mice, the loss of inner hair cells (IHCs) (Myo7A⁺) was observed (indicated by white arrows), and the administration of IP6 (80 mg/kg) or 2% IP6 through drinking water moderately mitigated the loss of IHCs in the base regions of the cochlear tissues of mice.

From the above, it can be clearly seen that IP6, fucoxanthin or the combination thereof provided in the present disclosure may improve the loss of HCs and the hearing thresholds, and thus can be useful for rescuing the hearing loss caused by chemotherapy, antibiotic, or aging, thereby improving the quality of life of patients with cancer or infection and the elderly.

It is obvious to a person skilled in the art that with the advancement of technology, the basic idea may be implemented in various ways. The embodiments are thus not limited to the examples described above; instead, they may vary within the scope of the claims.

The embodiments described hereinbefore may be used in any combination with each other. Several of the embodiments may be combined together to form a further embodiment. A compound, a composition or a method, disclosed herein, may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages.

### REFERENCES

[1] Saad N., Esa N., Ithnin H., et al., Optimization of optimum condition for phytic acid extraction from rice bran. African Journal of Plant Science 2011; 5 (3): 168-176.
[2] Lehrfeld, J., High-performance liquid chromatography analysis of phytic acid on a pH-stable, macroporous polymer column. Cereal Chemistry 1989; 66 (6): 510-515.
[3] Wu X., Li X., Song Y., et al., Allicin protects auditory hair cells and spiral ganglion neurons from cisplatin-Induced Apoptosis. Neuropharmacology 2017; 116:429-440.
[4] Dong Y., Guo C. R., Chen D., et al., Association between age-related hearing loss and cognitive decline in C57BL/6J mice. Molecular Medicine Reports 2018; 18:1726-1732.
[5] Neal C., Kennon-McGill S., Freemyer A., et al., Hair cell counts in a rat model of sound damage: Effects of tissue preparation & identification of regions of hair cell loss. Hearing Research 2015; 328:120-132.
[6] Ninoyu Y., Sakaguchi H., Lin C., et al., The integrity of cochlear hair cells is established and maintained through the localization of Dial at apical junctional complexes and stereocilia. Cell Death and Disease 2020; 11:536.
[7] Akil O., Oursler A. E., Fan K., et al., Mouse auditory brainstem response testing. Bio-protocol 2016; 6: e1768.
[8] Ingham N. J., Pearson S., Steel K. P., Using the auditory brainstem response (ABR) to determine sensitivity of hearing in mutant mice. Current Protocols in Mouse Biology 2011; 1:279-287.
[9] Choi M. Y., Yeo S. W., Park K. H., Hearing restoration in a deaf animal model with intravenous transplantation of mesenchymal stem cells derived from human umbilical cord blood. Biochemical and Biophysical Research Communications 2012; 427:629-636.
[10] Tsai S. C., Lin F. C., Chang K. H., et al., The intravenous administration of skin-derived mesenchymal stem cells ameliorates hearing loss and preserves cochlear hair cells in cisplatin-injected mice: SMSCs ameliorate hearing loss and preserve outer hair cells in mice. Hearing Research. 2022; 413:108254.
[11] Tsai S. C., Yang K. D., Chang K. H., et al., Umbilical cord mesenchymal stromal cell-derived exosomes rescue the loss of outer hair cells and repair cochlear damage in cisplatin-injected mice. International Journal of Molecular Sciences. 2021; 22 (13): 6664.
[12] Rubino S. J., Mayo L., Wimmer I., et al., Acute microglia ablation induces neurodegeneration in the somatosensory system. Nature Communications. 2018; 9 (1): 4578.
[13] Spangenberg E., Severson P. L., Hohsfield L. A., et al. Sustained microglial depletion with CSFIR inhibitor impairs parenchymal plaque development in an Alzheimer's disease model. Nature Communications. 2019; 10 (1): 3758.

What is claimed is:

1. A method for preventing or treating hearing loss in a subject in need thereof, comprising administering an effective amount of a composition to the subject, wherein the composition comprises at least one of inositol hexaphosphate or a salt or derivative thereof and fucoxanthin or a derivative thereof, and at least one of a pharmaceutically acceptable excipient thereof and a food excipient thereof.

2. The method according to claim 1, wherein the hearing loss is congenital or acquired.

3. The method according to claim 1, wherein the hearing loss is caused by at least one of infection, noise exposure, chemotherapy, antibiotic, and aging.

4. The method according to claim 1, wherein the subject suffers from the hearing loss involving degeneration or loss of hair cells.

5. The method according to claim 4, wherein the hair cells are vestibular hair cells, cochlear hair cells, or a combination thereof.

6. The method according to claim 1, wherein the subject suffers from at least one of sensorineural hearing loss, tinnitus and vertigo.

7. The method according to claim 1, wherein the subject suffers from acute hearing loss, chronic hearing loss, primary tinnitus, Meniere's disease, or labyrinthine concussion.

8. The method according to claim 1, wherein the salt or derivative of the inositol hexaphosphate is selected from the group consisting of sodium phytate, potassium phytate, calcium phytate, dipotassium phytate, calcium-magnesium phytate, magnesium phytate, and sodium phytate dodecahydrate.

9. The method according to claim 1, wherein the composition comprises the inositol hexaphosphate or the salt or derivative thereof without the fucoxanthin or the derivative thereof, and the inositol hexaphosphate or the salt or derivative thereof serves as a sole active ingredient for preventing or treating the hearing loss in the composition.

10. The method according to claim 1, wherein the composition comprises the fucoxanthin or the derivative thereof without the inositol hexaphosphate or the salt or derivative thereof, and the fucoxanthin or the derivative thereof serves as a sole active ingredient for preventing or treating the hearing loss in the composition.

11. The method according to claim 1, wherein the composition comprises the inositol hexaphosphate or the salt or derivative thereof and the fucoxanthin or the derivative thereof.

12. The method according to claim 1, wherein the inositol hexaphosphate or the salt or derivative thereof or the fucoxanthin or the derivative thereof is present in an amount of from 0.1% to 60% by weight of the composition.

13. The method according to claim 1, wherein the inositol hexaphosphate or the salt or derivative thereof or the fucoxanthin or the derivative thereof is administered to the subject in an effective amount of from about 1 mg/kg/day to about 500 mg/kg/day.

14. The method according to claim 13, wherein the inositol hexaphosphate or the salt or derivative thereof or the fucoxanthin or the derivative thereof is administered to the subject in an effective amount of from about 3 mg/kg/day to about 80 mg/kg/day.

15. The method according to claim 1, wherein the composition is administered to the subject orally, intravenously, subcutaneously, intradermally, intrathecally, intraperitoneally, intranasally, intramuscularly, intrapleuraly, topically, or through nebulization.

16. The method according to claim 1, wherein the composition is a pharmaceutical composition or an edible composition.

17. The method according to claim 16, wherein the edible composition comprises at least one of soy, barley, corn, millet, rice bran, rice germ, wheat, buckwheat, fair groat, roasted groat, oak, quinoa seed, seaweed, and an extract thereof.

18. The method according to claim 1, wherein the composition is in a form selected from the group consisting of an injection formulation, a dry powder, a tablet, an oral liquid, a flake, a film, a lozenge, a capsule, a granule, a pill, a gel, a lotion, an ointment, an emulsifier, a paste, a cream, an eye drop, and a salve.

19. The method according to claim 1, wherein the pharmaceutically acceptable excipient or the food excipient is selected from the group consisting of a filler, a binder, a preservative, a disintegrating agent, a lubricant, a suspending agent, a wetting agent, a solvent, a surfactant, an acid, a flavoring agent, polyethylene glycol (PEG), alkylene glycol, sebacic acid, dimethyl sulfoxide, an alcohol, calcium stearate, microcrystalline cellulose, silicon dioxide, gelatin, fat, glycerin, dietary fiber, alginate, pectin, carrageenan, amidated pectin, xanthan, gellan gum, karaya gum, rhamsan, welan, gum ghatti, gum arabic, and any combination thereof.

20. The method according to claim 1, wherein the composition is administered to the subject with a frequency selected from the group consisting of once per month, 2 times per month, 3 times per month, 4 times per month, 5 times per month, 6 times per month, 7 times per month, 8 times per month, once per week, 2 times per week, 3 times per week, 4 times per week, 5 times per week, 6 times per week, once every three days, 2 times every three days, 3 times every three days, once every two days, 2 times every two days, once per day, and 2 times per day.
